(19) United States
(12) Patent Application Publication
(10) Pub. No.: US 2025/0205403 A1
(43) Pub. Date: Jun. 26, 2025

(54) DIALYSIS SYSTEM HAVING PUMP ACTUATOR POSITION DETECTION

(71) Applicants: Baxter International Inc., Deerfield, IL (US); Baxter Healthcare SA, Glattpark (Opfikon) (CH)

(72) Inventors: Oskar Erik Frode Styrbjörn FÄLLMAN, Lund (SE); Michael PETTERSSON, MALMÖ (SE); Sverre Haftor KNUTSEN, MALMÖ (SE)

(21) Appl. No.: 18/392,196

(22) Filed: Dec. 21, 2023

(51) Int. Cl. A61M 1/28

(52) U.S. Cl. CPC A61M 1/282; A61M 1/288; A61M 2205/15; A61M 2205/3331; A61M 2205/3334

(57) ABSTRACT

A peritoneal dialysis (“PD”) system includes a PD fluid pump including a reciprocating member having a home position; at least one pressure sensor positioned and arranged to sense pressure PD fluid pumped by the PD fluid pump; and a control unit configured to control the PD fluid pump and to take PD fluid pressure readings from the at least one pressure sensor, the control unit further configured to use the PD fluid pressure readings to determine when the reciprocating member is in the home position, and to stop the PD fluid pump when the reciprocating member is in the home position.

### TECHNICAL FIELD

The present disclosure relates generally to medical fluid treatments, and in particular to dialysis fluid treatments that require fluid heating.

### BACKGROUND

Due to various causes, a person's renal system can fail. Renal failure produces several physiological derangements. It is no longer possible to balance water and minerals or to excrete daily metabolic load. Toxic end products of metabolism, such as, urea, creatinine, uric acid, and others may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works, or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. Automated PD machines, however, perform the cycles automatically, typically while the patient sleeps. The PD machines free patients from having to manually perform the treatment cycles and from having to transport supplies during the day. The PD machines connect fluidly to an implanted catheter, to a source or bag of fresh dialysis fluid and to a fluid drain. The PD machines pump fresh dialysis fluid from a dialysis fluid source, through the catheter and into the patient's peritoneal chamber. The PD machines also allow for the dialysis fluid to dwell within the chamber and for the transfer of waste, toxins and excess water to take place. The source may include multiple liters of dialysis fluid including several solution bags.

The PD machines pump used or spent dialysate from the patient's peritoneal cavity, though the catheter, to a drain. As with the manual process, several drain, fill, and dwell cycles occur during dialysis. A “last fill” may occur at the end of an APD treatment. The last fill fluid may remain in the peritoneal chamber of the patient until the start of the next treatment, or may be manually emptied at some point during the day.

In any of the above modalities using an automated machine, component expense, calibration, and maintenance are key design considerations. If a component can be eliminated, not only is its cost eliminated, but potential calibration of the component is eliminated along with its maintenance and/or replacement. Component removal reduces machine weight and also frees space within the machine housing or allows the housing to be made smaller.

For each of the above reasons, it is desirable to provide an APD machine that reduces component expense, calibration, and/or maintenance.

### SUMMARY

The present disclosure sets forth an automated peritoneal dialysis (“PD”) system, which includes a PD machine or cycler. The PD machine is capable of delivering fresh, heated PD fluid to the patient at, for example, 14 kPa (2.0 psig) or higher. The PD machine is capable of removing used PD fluid or effluent from the patient at, for example, between −5 kPa (−0.73 psig) and −15 kPa (−2.2 psig), such as −9 kPa (−1.3 psig) or higher. Fresh PD fluid may be delivered via a dual lumen patient line to the patient and is first heated to body fluid temperature, e.g., 37° C. The heated PD fluid is then pumped through a fresh PD fluid lumen of the dual lumen patient line into a disposable filter set, which is connected to the patient's transfer set, which is in turn connected to an indwelling catheter leading into the patient's peritoneal cavity. The disposable filter set communicates fluidly with the fresh and used PD fluid lumens of the dual lumen patient line. The disposable filter set is provided in one embodiment as a last chance filter for the PD machine, which may be heat disinfected between treatments.

The system may include one or more PD fluid container or bag that supplies fresh PD fluid to the PD machine or cycler. The PD machine or cycler may include internal lines having two-way or three-way valves and at least one PD fluid pump for pumping fresh PD fluid from the one or more PD fluid container or bag to a patient and for removing used PD fluid from the patient to a house drain or drain container. One or more flexible PD fluid line leads from the PD machine or cylcer's internal lines to the one or more PD fluid container or bag. The flexible dual lumen patient line mentioned above leads from the PD machine or cylcer's internal lines to the patient. A flexible drain line leads from the PD machine or cylcer's internal lines to the house drain or drain container. The system in one embodiment disinfects all internal lines, the PD fluid lines and the dual lumen patient line after treatment for reuse in the next treatment. The disinfection may involve heat disinfection using leftover fresh PD fluid.

The PD machine or cycler also includes different types of sensors that output to a control unit of the machine. The different types of sensors include, for example, temperature sensors, pressure sensors, a leak detection sensor and possibly a flow sensor. The pressure sensors detect PD fluid pressures and are used to control the PD fluid pressures (negative and positive) caused by a PD fluid pump. Where the filter set and dual lumen patient line are provided, a filter membrane of the filter set causes a pressure drop in fresh PD fluid pressure. The pressure outputted by the pump to the filter membrane is accordingly greater than the pressure experienced by the patient downstream from the filter membrane due to the pressure drop. The PD fluid pressure downstream from the filter membrane is accordingly the important pressure to monitor for use as feedback to control the PD fluid pump, such that the PD fluid pressure experienced by the patient is at or below a patient PD fluid pressure limit.

One or more pressure sensor is located so as to sense the PD fluid pressure in the used PD fluid lumen of the dual lumen patient line, which is the important pressure downstream of the filter membrane. The pressure sensor positioned to sense the PD fluid pressure in the fresh PD fluid lumen is therefore not as critical and instead is used to sense, for example, kinks or obstructions is the fresh PD fluid lumen.

The PD fluid pump is, in one embodiment, a piston pump that includes a housing holding a cylinder within which a piston is actuated via a motor, under control of a motor driver (considered a part of the overall control unit), wherein the motor drives a motion coupler coupled to a piston. The motion coupler converts a rotational motion of the motor to a rotational and translational movement of the piston. The motion coupler moves the piston in and out relative to the cylinder to create positive and negative pumping pressure, respectively. The motion coupler also rotates the piston within the cylinder to move PD fluid from an inlet port to an outlet port.

The motor for the piston pump is in one embodiment a stepper motor. The system and associated methodology of the present disclosure apply in one implementation to where the pump piston position needs to be related to a relative position of the motor. External sensors, such as an absolute encoder operating with the stepper motor, allow the precise angular location of the motor shaft to be known, such that a particular angular location corresponding to a desired piston turning or home position is detectable. Here, rotating the shaft of the stepper motor to the precise angular location in turn places the pump piston at the desired piston turning or home position. The present system and method allows the desired piston turning or home position to be determined and set using an existing pressure sensor, negating the need for an external sensor, such as an absolute encoder.

The present system and its associated methodology include placing the PD fluid pump in a piston turning position (which may be referred to herein as a home position) in which a push partial stroke creating positive downstream pressure has just ended and a pull partial stroke pulling from an upstream side begins, which is recorded by one or more pressure sensor. It is desirable to place the PD fluid pump in the piston turning or home position at shutdown because dried residuals on the outside of the piston and and/or on the inside of the cylinder may make it hard to start the PD fluid pump after long periods of off time. In the piston turning or home position, the linear piston force is high because the ratio of a rotation of the pump or stepper motor to a linear movement of piston is low, such that the pump motor needs less motor torque to translate and rotate the piston within the cylinder when starting the PD fluid pump.

It is contemplated for control unit of the PD machine of the present system to determine the piston turning or home position in one of at least two operational modes, one mode in which PD fluid is pumped through and open fluid path and a second mode in which PD fluid is pumped through a closed fluid path, e.g., against one or more closed valve. In the open fluid path operational mode, even though the fluid path is open, there is some fluid resistance due to line or tube size, line surface friction and possibly battling head height. The pressure downstream from the PD fluid pump varies accordingly with the movement of piston within the cylinder. The control unit is programmed to extract components or features from the pressure signal that correspond to a position of the pump piston, wherein a certain repeatable portion of the components or features of the pressure signal correspond to the piston turning or home position.

In the closed fluid path operational mode, e.g., pumping against one or more closed valve, the fluid pressure will have an approximate linear relationship with the movement of the piston within the cylinder. The closed fluid path operational mode is implemented in one embodiment at slow speeds for the PD fluid pump, allowing the control unit to determine when a derivative of the pressure signal from one or more pressure sensor decreases to below zero (zero crossing), which corresponds to the piston turning or home position. Compared to the open fluid path operational mode, the closed fluid path operational mode requires a period of time in which treatment is not being performed, e.g., any idle time of the PD machine, such as during a patient dwell, or perhaps after treatment. The closed fluid path operational mode achieves a high accuracy by using a slow actuation of the PD fluid pump together with a high sample rate taken by the processor for reading from one or more pressure sensor. The closed fluid path operational mode, in one embodiment, evaluates only a single full stroke (e.g., a single partial push and a single partial pull stroke), such that it is necessary to take an adequate amount of pressure data points over the single full stroke.

The control unit may combine the results from the open and closed fluid path operational modes to increase reliability in finding the piston turning or home position. Generally, the closed fluid path mode is more accurate, but the open fluid path mode confirming the closed fluid path piston turning or home position finding provides redundancy and increases reliability.

Aspects of the subject matter described herein may be useful alone or in combination with one or more other aspect described herein. Without limiting the foregoing description, in a first aspect of the present disclosure, which may be combined with any other aspect, or portion thereof, a peritoneal dialysis (“PD”) system includes a PD fluid pump including a reciprocating member having a home position, at least one pressure sensor positioned and arranged to sense the pressure of PD fluid pumped by the PD fluid pump, and a control unit configured to control the PD fluid pump and to take PD fluid pressure readings from the at least one pressure sensor. The control unit is further configured to use the PD fluid pressure readings to determine when the reciprocating member is in the home position, and when desired, to stop the PD fluid pump when the reciprocating member is in the home position.

In a second aspect of the present disclosure, which may be combined with any other aspect, or portion thereof, the at least one pressure sensor is positioned and arranged to take the pressure of PD fluid along a line downstream of the PD fluid pump.

In a third aspect of the present disclosure, which may be combined with any other aspect, or portion thereof, the at least one pressure sensor is positioned and arranged to take the pressure of PD fluid along an open fluid line, wherein the pressure of PD fluid changes in a pattern according to movement of the reciprocating member, and wherein a feature in the pattern is related the home position.

In a fourth aspect of the present disclosure, which may be combined with any other aspect, or portion thereof, the open fluid line is used during a PD treatment, the at least one pressure sensor taking the pressure of PD fluid during the PD treatment.

In a fifth aspect of the present disclosure, which may be combined with any other aspect, or portion thereof, the control unit is configured to take PD fluid pressure readings from the at least one pressure sensor at a sample rate of 50 to 500 Hz.

In a sixth aspect of the present disclosure, which may be combined with any other aspect, or portion thereof, the at least one pressure sensor is positioned and arranged to take the pressure of PD fluid along a closed fluid line, wherein the control unit is configured to use at least one algorithm to analyze a derivative of a change of the pressure of PD fluid over a full stroke of the PD fluid pump, and wherein a zero crossing of the derivative of the change of the pressure of PD fluid over the stroke corresponds to the home position.

In a seventh aspect of the present disclosure, which may be combined with any other aspect, or portion thereof, the closed fluid line is closed when the system is in an idle state, wherein the control unit uses the at least one algorithm.

In an eighth aspect of the present disclosure, which may be combined with any other aspect, or portion thereof, the control unit is configured to take PD fluid pressure readings from the at least one pressure sensor at a sample rate of 50 to 200 Hz while the control unit uses at least one algorithm.

In a ninth aspect of the present disclosure, which may be combined with any other aspect, or portion thereof, the control unit is configured to cause the PD fluid pump to pump at a rate of 0.1 to 10 rpm while the control unit uses at least one algorithm.

In a tenth aspect of the present disclosure, which may be combined with any other aspect, or portion thereof, the at least one algorithm includes a recursive estimation algorithm, optionally a recursive least squares (“RLS”) algorithm or a linear quadratic estimator (“LQE”) algorithm.

In an eleventh aspect of the present disclosure, which may be combined with any other aspect, or portion thereof, the at least one pressure sensor includes a plurality of pressure sensors positioned and arranged to take the pressure of PD fluid along the closed fluid line, and wherein the at least one algorithm employs a linear quadratic estimation (“LQE”) filter.

In a twelfth aspect of the present disclosure, which may be combined with any other aspect, or portion thereof, the PD fluid pump is a piston pump and the reciprocating member includes a piston moving within a cylinder.

In a thirteenth aspect of the present disclosure, which may be combined with any other aspect, or portion thereof, the home position corresponds to a distal end of the piston being closest to an end wall of the cylinder.

In a fourteenth aspect of the present disclosure, which may be combined with any other aspect, or portion thereof, a peritoneal dialysis (“PD”) system includes a PD fluid pump including a reciprocating member having a home position and at least one pressure sensor positioned and arranged along an open fluid line to sense the pressure of PD fluid pumped by the PD fluid pump, where the pressure of PD fluid changes in a pattern according to movement of the reciprocating member, The PD system also includes a control unit configured to control the PD fluid pump and to take PD fluid pressure readings from the at least one pressure sensor. The control unit is further configured to use a feature in the pattern to determine when the reciprocating member is in the home position.

In a fifteenth aspect of the present disclosure, which may be combined with any other aspect, or portion thereof, the feature includes a minimum pressure in the PD fluid pressure pattern.

In a sixteenth aspect of the present disclosure, which may be combined with any other aspect, or portion thereof, a peritoneal dialysis (“PD”) system includes a PD fluid pump including a reciprocating member having a home position and at least one pressure sensor positioned and arranged along a closed fluid line to sense the pressure of PD fluid pumped by the PD fluid pump. The PD system also includes a control unit configured to use at least one algorithm to analyze a derivative of the pressure of PD fluid over a push partial stroke or a pull partial stroke of the PD fluid pump, where a zero crossing of the derivative of the change of the pressure of PD fluid over the stroke corresponds to the home position.

In a seventeenth aspect of the present disclosure, which may be combined with any other aspect, or portion thereof, the at least one algorithm includes a recursive estimation algorithm, optionally a recursive least squares (“RLS”) algorithm or a linear quadratic estimator (“LQE”) algorithm.

In an eighteenth aspect of the present disclosure, which may be combined with any other aspect, or portion thereof, the at least one pressure sensor includes a plurality of pressure sensors positioned and arranged to take the pressure of PD fluid along the closed fluid line, and wherein the at least one algorithm employs a linear quadratic estimation (“LQE”) filter.

In a nineteenth aspect of the present disclosure, which may be combined with any other aspect, or portion thereof, any of the features, functionality and alternatives described in connection with any one or more of FIGS. 1 to 7 may be combined with any of the features, functionality and alternatives described in connection with any other of FIGS. 1 to 7.

In light of the above aspects and present disclosure set forth herein, it is an advantage of the present disclosure to provide a PD system and associated methodology that uses a pressure sensor output to determine a pump actuator position.

It is another advantage of the present disclosure to provide a PD system and associated methodology that determines a pump actuator position without requiring a dedicated pump actuator position sensor.

It is a further advantage of the present disclosure to provide a PD system and associated methodology that is able to set a pump actuator at the end of use such that it is in an optimal position for starting upon a next use.

It is a yet another advantage of the present disclosure to provide a PD system and associated methodology that is able to set a pump actuator at the end of use such that it is easier to start upon a next use.

It is yet a further advantage of the present disclosure to provide a PD system and associated methodology that improves motor angular position accuracy and thus fluid volume pumped accuracy, such as fine dosing of an additive fluid.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the improvements or advantages listed herein, and it is expressly contemplated to claim individual advantageous embodiments separately. In particular, the system of the present disclosure may have any one or more or all of the drip prevention structure and methodology, PD fluid container emptying structure and methodology and patient connection before drain check structure and methodology described herein. Moreover, it should be noted that the language used in the specification has been selected principally for readability and instructional purposes, and not to limit the scope of the inventive subject matter.

### BRIEF DESCRIPTION OF THE FIGURES

FIG. 1 is a fluid flow schematic of one embodiment for a PD system in a treatment state, according to an example embodiment of the present disclosure.

FIG. 2 is a fluid flow schematic of one embodiment for a PD system in a disinfection state, according to an example embodiment of the present disclosure.

FIG. 3 is a partially sectioned elevation view of one embodiment for a medical fluid, e.g., PD fluid, pump suitable for use in the system and associated methodologies of the present disclosure.

FIG. 4 is a set of plots relating pump motor position, downstream PD fluid pressure, and downstream PD fluid flowrate.

FIG. 5 is a set of plots relating pump motor frequency, pump motor position using a tachometer output for comparison, pump motor position using minimum pressure of the open fluid path operational mode, and pump motor position using minimum pressure of the open fluid path operational mode compensated for position aliasing.

FIGS. 6 and 7 are a set of plots relating pressure buildup during a full stroke of the PD fluid pumps and a derivative of the pressure buildup during the full stroke of the PD fluid pumps pursuant to the closed fluid path operational mode.

### DETAILED DESCRIPTION

### System Overview

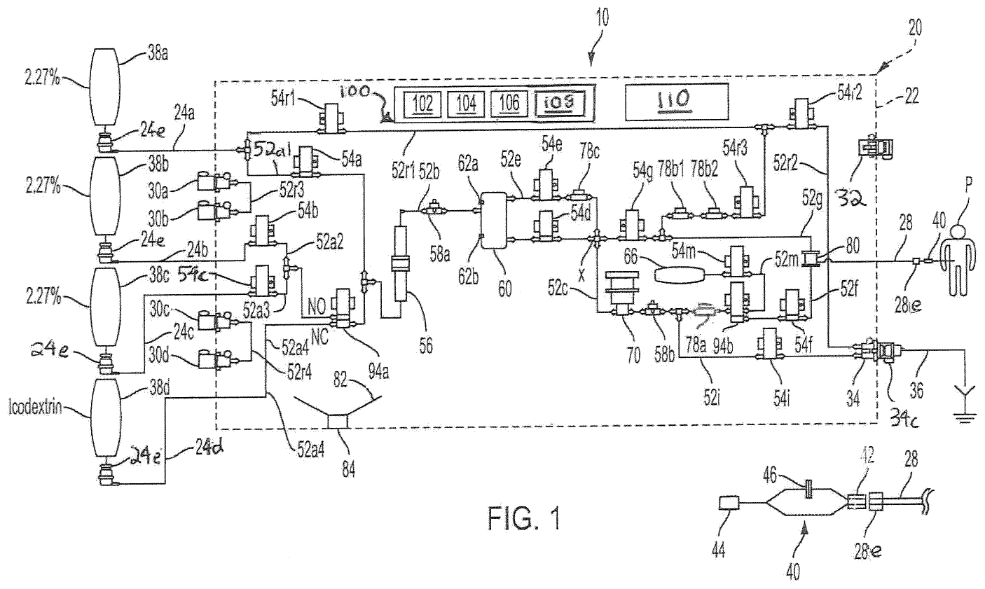

Referring now to the drawings and in particular to FIG. 1, a medical system having the pump actuator position detection of the present disclosure is illustrated via peritoneal dialysis (“PD”) system 10. System 10 includes a PD machine or cycler 20 and a control unit 100 including one or more processor 102, one or more memory 104, video controller 106, a motor driver 108 and user interface 110. User interface 110 may alternatively or additionally be a remote user interface, e.g., via a tablet or smartphone. Control unit 100 may also include a transceiver and a wired or wireless connection to a network (not illustrated), e.g., the internet, for sending treatment data to and receiving prescription instructions/changes from a doctor's or clinician's server interfacing with a doctor's or clinician's computer. Control unit 100 in an embodiment controls all electrical fluid flow and heating components of system 10 and receives outputs from all sensors of system 10. System 10 in the illustrated embodiment includes durable and reusable components that contact fresh and used PD fluid, for example, which necessitates that PD machine or cycler 20 be disinfected between treatments, e.g., via heat disinfection.

System 10 in FIG. 1 includes an inline resistive heater 56, reusable supply lines or tubes 52al to 52a4 and 52b, air trap 60 operating with respective upper and lower level sensors 62a and 62b, air trap valve 54d, vent valve 54e located along vent line 52e, reusable line or tubing 52c, PD fluid pump 70, temperature sensors 58a and 58b, pressure sensors 78a, 78b1 or 78b2, reusable patient tubing or lines 52f and 52g having respective valves 54f and 54g, dual lumen patient line 28, a hose reel 80 for retracting patient line 28, reusable drain tubing or line 52i extending to drain line connector 34 and having a drain line valve 54i, and reusable recirculation disinfection tubing or lines 52r1 and 52r2 operating with respective disinfection valves 54r1 and 54r2. A third recirculation or disinfection tubing or line 52r3 extends between disinfection or PD fluid line connectors 30a and 30b for use during disinfection. A fourth recirculation or disinfection tubing or line 52r4 extends between disinfection connectors 30c and 30d for use during disinfection.

System 10 also includes PD fluid containers or bags 38a to 38c (e.g., holding the same or different formulations of PD fluid), which connect to distal ends 24e of reusable PD fluid lines 24a to 24c, respectively. System 10 further includes a fourth PD fluid container or bag 38d that connects to a distal end 24e of reusable PD fluid line 24d. Fourth PD fluid container or bag 38d may hold the same or different type (e.g., icodextrin) of PD fluid than provided in PD fluid containers or bags 38a to 38c. Reusable PD fluid lines 24a to 24d extend in one embodiment through apertures (not illustrated) defined or provided by housing 22 of PD machine 20.

System 10 in the illustrated embodiment includes four disinfection or PD fluid line connectors 30a to 30d for connecting to distal ends 24e of reusable PD fluid lines 24a to 24d, respectively, during disinfection. System 10 also provides a patient line connector 32 that includes an internal lumen, e.g., a U-shaped lumen, which for disinfection directs fresh or used dialysis fluid from one PD fluid lumen of a connected distal end 28e of dual lumen patient line 28 into the other PD fluid lumen. Reusable supply tubing or lines 52al to 52a4 communicate with reusable supply lines 24a to 24d, respectively. Reusable supply tubing or lines 52al to 52a3 operate with valves 54a to 54c, respectively, to allow PD fluid from a desired PD fluid container or bag 38a to 38c to be pulled into PD machine 20. Three-way valve 94a in the illustrated example allows for control unit 100 to select between (i) 2.27% (or other) glucose dialysis fluid from container or bag 38b or 38c and (ii) icodextrin from container or bag 38d. In the illustrated embodiment, icodextrin from container or bag 38d is connected to the normally closed port of three-way valve 94a.

System 10 is constructed in one embodiment such that drain line 52i during a patient fill is fluidly connected downstream from PD fluid pump 70. In this manner, if drain valve 54i fails or somehow leaks during the patient fill of patient P, fresh PD fluid is pushed down disposable drain line 36 instead of used PD fluid potentially being pulled into pump 70. Disposable drain line 36 is in one embodiment removed for disinfection, wherein drain line connector 34 is capped via a cap 34c to form a closed disinfection loop. PD fluid pump 70 may be an inherently accurate pump, such as a piston pump, or less accurate pump, such as a gear pump that operates in cooperation with a flowmeter (not illustrated) to control fresh and used PD fluid flowrate and volume.

System 10 may further include a leak detection pan 82 located at the bottom of housing 22 of PD machine 20 and a corresponding leak detection sensor 84 outputting to control unit 100. In the illustrated example, system 10 is provided with an additional pressure sensor 78c located upstream of PD fluid pump 70, which allows for the measurement of the suction pressure of pump 70 to help control unit 100 more accurately determine pump volume. Additional pressure sensor 78c in the illustrated embodiment is located along vent line 52e, which may be filled with air or a mixture of air and PD fluid, but which should nevertheless be at the same negative pressure as PD fluid located within PD fluid line 52c.

System 10 in the example of FIG. 1 includes redundant pressure sensors 78b1 and 78b2, the output of one of which is used for pump control, while the output of the other pressure sensor is a safety or watchdog output to make sure the control pressure sensor is reading accurately. Pressure sensors 78b1 and 78b2 are located along a line including a third recirculation valve 54r3. System 10 may further employ one or more cross, marked via an X in FIG. 1, which may (i) reduce the overall amount and volume of the internal, reusable tubing, (ii) reduce the number of valves needed, and (iii) allow the portion of the fluid circuitry shared by both fresh and used PD fluid to be minimized.

System 10 in the example of FIG. 1 further includes a source of acid, such as a citric acid container or bag 66. Citric acid container or bag 66 is in selective fluid communication with second three-way valve 94b via a citric acid valve 54m located along a citric acid line 52m. Citric acid line 52m is connected in one embodiment to the normally closed port of second three-way valve 94b, so as to provide redundant valves between citric acid container or bag 66 and the PD fluid circuit during treatment. The redundant valves ensure that no citric (or other) acid reaches the treatment fluid lines during treatment. Citric (or other) acid is used instead during disinfection.

Control unit 100 in an embodiment uses feedback from any one or more of pressure sensors 78b1 or 78b2 to enable PD machine 20 to deliver fresh, heated PD fluid to the patient at, for example, 14 kPa (2.0 psig) or higher. The pressure feedback is used to enable PD machine 20 to remove used PD fluid or effluent from the patient at, for example, between −5 kPa (−0.73 psig) and −15 kPa (−2.2 psig), such as −9 kPa (−1.3 psig) or higher (more negative). The pressure feedback may be used in a proportional, integral, derivative (“PID”) pressure routine for pumping fresh and used PD fluid at a desired positive or negative pressure.

Inline resistive heater 56 under control of control unit 100 is capable of heating fresh PD fluid to body temperature, e.g., 37° C., for delivery to patient P at a desired flowrate. Control unit 100 in an embodiment uses feedback from temperature sensor 58a in a PID temperature routine for pumping fresh PD fluid to patient P at a desired temperature.

FIG. 1 also illustrates that system 10 includes and uses a disposable filter set 40, which communicates fluidly with the fresh and used PD fluid lumens of dual lumen patient line 28. Disposable filter set 40 includes a disposable connector 42 that connects to a distal end 28e of reusable patient line 28. Disposable filter set 40 also includes a connector 44 that connects to the patient's transfer set. Disposable filter set 40 further includes a sterilizing grade hydrophilic filter membrane 46 that further filters fresh PD fluid. Disposable filter set 40 is provided in one embodiment as a last chance filter for PD machine 20, which has been heat disinfected between treatments. Any pathogens that may remain after disinfection, albeit unlikely, are filtered from the PD fluid via the hydrophilic membrane 46 of disposable filter set 40.

FIG. 1 illustrates system 10 setup for treatment with PD fluid containers or bags 38a to 38d connected via reusable, flexible PD fluid lines 24a to 24d, respectively. Dual lumen patient line 28 is connected to patient P via disposable filter set 40. Disposable drain line 36 is connected to drain line connector 34. In FIG. 1, PD machine or cycler 20 of system 10 is configured to perform multiple patient drains, patient fills, patient dwells, and a priming procedure, as part of or in preparation for treatment.

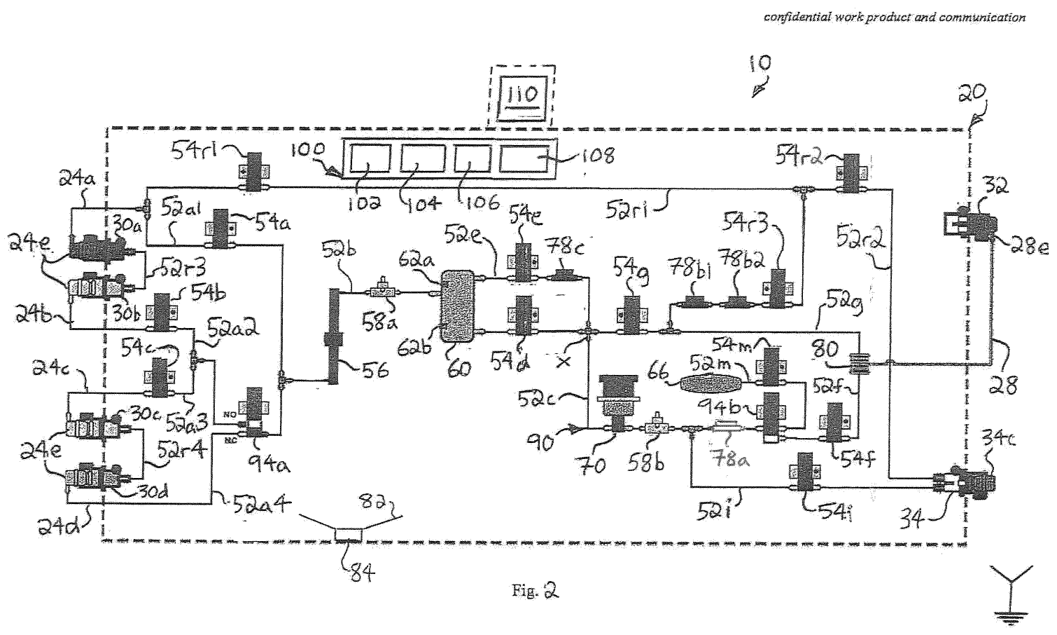

FIG. 2 illustrates system 10 in a disinfection mode. PD fluid containers or bags 38a to 38d are removed and flexible PD fluid lines 24a to 24d are plugged instead in a sealed manner into disinfection or PD fluid line connectors 30a to 30d, respectively. Reusable Dual lumen patient line 28 is disconnected from disposable filter set 40 (which is discarded), and distal end 28e of dual lumen patient line 28 is plugged sealingly into patient line connector 32. Disposable drain line 36 is removed from drain line connector 34 and discarded. Drain line connector 34 is capped via cap 34c to form a closed disinfection loop 90. PD machine or cycler 20 of system 10 in FIG. 2 is configured to perform a disinfection sequence, e.g., a heat disinfection sequence, in which fresh PD fluid is heated via inline heater 56 to a disinfection temperature, e.g., 70° C. to 90° C. PD fluid pump 70 circulates the heated PD fluid closed disinfection loop 90 for an amount of time needed to properly disinfect the fluid components and lines of the disinfection loop.

Hydrophilic filter membrane 46 of filter set 40 causes a pressure drop in fresh PD fluid pressure. The pressure outputted by PD fluid pump 70 through the fresh PD fluid lumen of dual lumen patient line 28 to hydrophilic filter membrane 46 is accordingly greater than the pressure experienced by patient P downstream from hydrophilic filter membrane 46 due to the pressure drop. The PD fluid pressure downstream from hydrophilic filter membrane 46 is accordingly the important pressure to monitor for use as feedback to control the PD fluid pump 70, such that the PD fluid pressure experienced by patient P is at or below the patient PD fluid pressure limits listed above.

Pressure sensors 78bl, 78b2 are located so as to sense the PD fluid pressure in the used PD fluid lumen of dual lumen patient line 28, which is the important pressure downstream of the filter membrane. A pressure sensor 78a positioned to sense the PD fluid pressure in the fresh PD fluid lumen is therefore not as critical and instead is used to sense, for example, kinks or obstructions is the fresh PD fluid lumen. It is accordingly contemplated to eliminate pressure sensor 78a positioned to sense the PD fluid pressure in the fresh PD fluid lumen and to instead estimate the PD fluid pressure using an output signal provided by motor driver 108 for the motor (e.g., stepper motor) used to drive PD fluid pump 70.

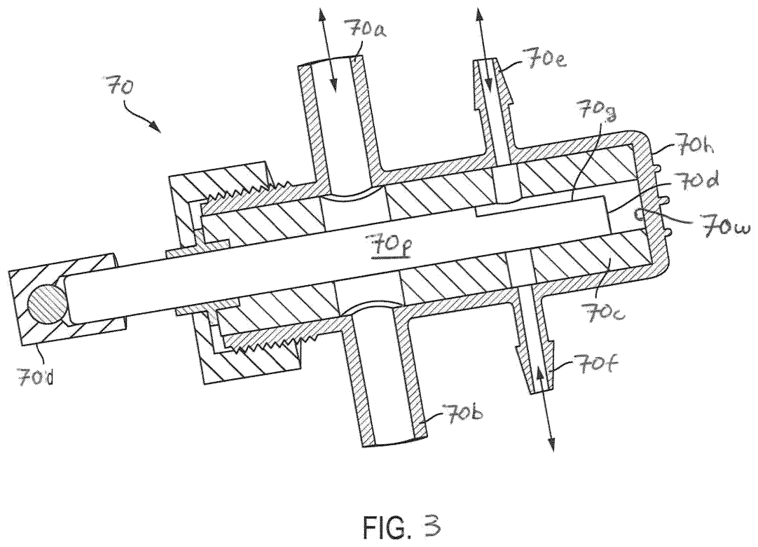

FIG. 3 illustrates that PD fluid pump 70 is in one embodiment a piston pump.

Piston pump 70 in the illustrated embodiment includes a housing 70h holding a cylinder 70c within which a piston 70p is actuated via a pump motor (e.g., stepper motor, not illustrated), driven by motor driver 108, which is under control of control unit 108, driving a motion coupler 70m coupled to piston 70p, wherein motion coupler 70m converts a rotational motion of the motor to a rotational and translational movement of piston 70p. Housing 70h includes fluid inlet/outlet ports 70e and 70f (bidirectional) and flush flow ports 70a and 70b (bidirectional or stagnant).

Motion coupler 70m moves piston 70p in and out relative to cylinder 70c to create positive and negative pumping pressure, respectively. Motion coupler 70m also rotates piston 70p within cylinder 70c to move fluid from one of ports 70e and 70f, acting as a PD or other fluid inlet port, to the other of ports 70e and 70f, acting as a PD or other fluid outlet port. The distal end of piston 70p includes a cutout or groove 70g forming a flat. The open area formed by groove 70g accepts PD or other fluid at the inlet port 70e or 70f (under negative pressure when piston 70p is retracted within cylinder 70c) and is then rotated to deliver PD fluid at the outlet port 70e or 70f (under positive pressure when piston 70p is extended within cylinder 70c). Groove 70g provides the valve functionality so that dialysis fluid pump 70 can have different flow directions.

The translational and rotational movement of piston 70p within cylinder 70c creates heat and friction. A flush flow of fluid may be provided accordingly to lubricate the translational and rotational movement of piston 70p within cylinder 70c. The flush flow of fluid, e.g., reverse osmosis, distilled or deionized water, is provided at flush flow ports 70a and 70b to contact piston 70p as it is moved translationally and rotationally within cylinder 70c. The flush flow of fluid may be circulated or stagnant.

### Pump Actuator Position Detection

System 10 and its associated methodology include placing PD fluid pump 70 in a piston turning position (which may be referred to herein as a home position) in which a push partial stroke (e.g., a compression stroke) creating positive downstream pressure has just ended and a partial stroke (e.g., a suction stroke) pulling from an upstream side begins, which is recorded by one or more pressure sensor 78a, 78b1, 78b2, 78c. In FIG. 3, the piston turning position or home occurs when the distal end 70d of piston 70p contacts, or comes closest to contacting, an inner surface of an end wall 70w of housing 70h. It is desirable to place PD fluid pump 70 in the piston turning or home position at shutdown because dried residuals on the outside of piston 70p and and/or on the inside of housing 70h may make it hard to start PD fluid pump 70 after long periods of off time. In the piston turning or home position, the linear piston force is high because the ratio of a rotation of the pump or stepper motor to a linear movement of piston 70p is low because a theoretical velocity of piston 70p is zero at the home position, while the pump motor rotation is non-zero. The stepper motor will accordingly need less motor torque to translate and rotate piston 70p within housing 70h when starting PD fluid pump 70 from the piston turning or home position, which is set at a prior pump shutdown.

It is possible to detect the piston turning or home position using a separate dedicated sensor, such as a tachometer, encoder or optical, magnetic or angle sensor. Such sensors however add cost, complexity and may involve an invasive attachment to the pump motor or pump housing 70h. System 10 is instead able to detect when piston 70p is at the piston turning or home position within housing 70h using an output from downstream pressure sensor 78a or upstream pressure sensor 78c. Notably, where dual lumen patient line 28 is used, the downstream fluid pressure measured by downstream pressure sensor 78a will be transmitted back up the used patient line lumen and will thus be also measured by pressure sensors 78b1, 78b2. The pressures read by pressure sensors 78b1, 78b2 will be lower due to the pressure drop across hydrophilic filter membrane 46. The outputs from pressure sensors 78b1, 78b2 may still be taken into account however, e.g., in addition to the output from downstream pressure sensor 78a when the distal end 28e of dual lumen patient line 28 is connected to the patient line connector 32.

It is contemplated for control unit 100 to determine the piston turning or home position in one of at least two operational modes, one mode in which PD fluid is pumped through and open fluid path and a second mode in which PD fluid is pumped through a closed fluid path, e.g., against one or more closed valve. In the open fluid path operational mode, even though the fluid path is open, there is some fluid resistance due to line or tube size, line surface friction and possibly battling head height. The pressure downstream from PD fluid pump 70 varies accordingly with the movement of piston 70p within cylinder 70c. Control unit 100 is programmed to extract components or features from the pressure signal that correspond to a position of piston 70p, wherein a repeatable portion of the components or features from the pressure signal correspond to the piston turning or home position. The accuracy of the open fluid path operational mode is a function of the sample time of one or more pressure sensor 78a, 78b, 78b2, or 78c and the sample time of the relative motor position (e.g., steps taken by a step motor driving piston 70p), which provides a large amount of data points resulting in a reliable position estimation. The open fluid path operational mode may be implemented by control unit 100 while PD fluid pump 70 is used for treatment, e.g., patient draining or filling. Since the open fluid path method uses a large amount of measurement points to estimate the position, it is a reliable option.

In the closed fluid path operational mode, e.g., pumping against one or more closed valve, the fluid pressure will have an approximate linear relationship with the movement of piston 70p within cylinder 70c. The closed fluid path operational mode is implemented in one embodiment at slow speeds for PD fluid pump 70, e.g., 5 to 50 ml/min, allowing control unit 100 to determine when a derivative of the pressure signal from one or more pressure sensor 78a, 78b1 or 78b2 decreases to below zero (zero crossing), which corresponds to the piston turning or home position. In an analogy, pressure sensor 78c could be used when closing the upstream fluid path. Compared to the open fluid path operational mode, the closed fluid path operational mode requires a period of time in which treatment is not being performed, e.g., at any idle time for PD machine 20, such as during a patient dwell, or perhaps after treatment. The closed fluid path operational mode achieves a high accuracy by using a slow actuation of PD fluid pump 70 together with a high sample rate taken by processor 102 of control unit 100 of the reading from one or more pressure sensor 78a, 78c, 78b1, or 78b2. The closed fluid path operational mode in one embodiment evaluates only a single suction and compression stroke (e.g., a single partial push stroke and a single partial pull stroke), such that it is necessary to take an adequate amount of pressure data points over the single stroke. In other embodiments, only the single partial pull stroke (e.g., the suction stroke) or the single partial push stroke (e.g., the compression stroke) is evaluated.

In an embodiment, control unit 100 combines the results from the open and closed fluid path operational modes to increase reliability in finding the piston turning or home position. Generally, the closed fluid path mode is more accurate, but the open fluid path mode confirming the closed fluid path piston turning or home position finding provides redundancy and increases reliability.

### Open Fluid Path Operational Mode

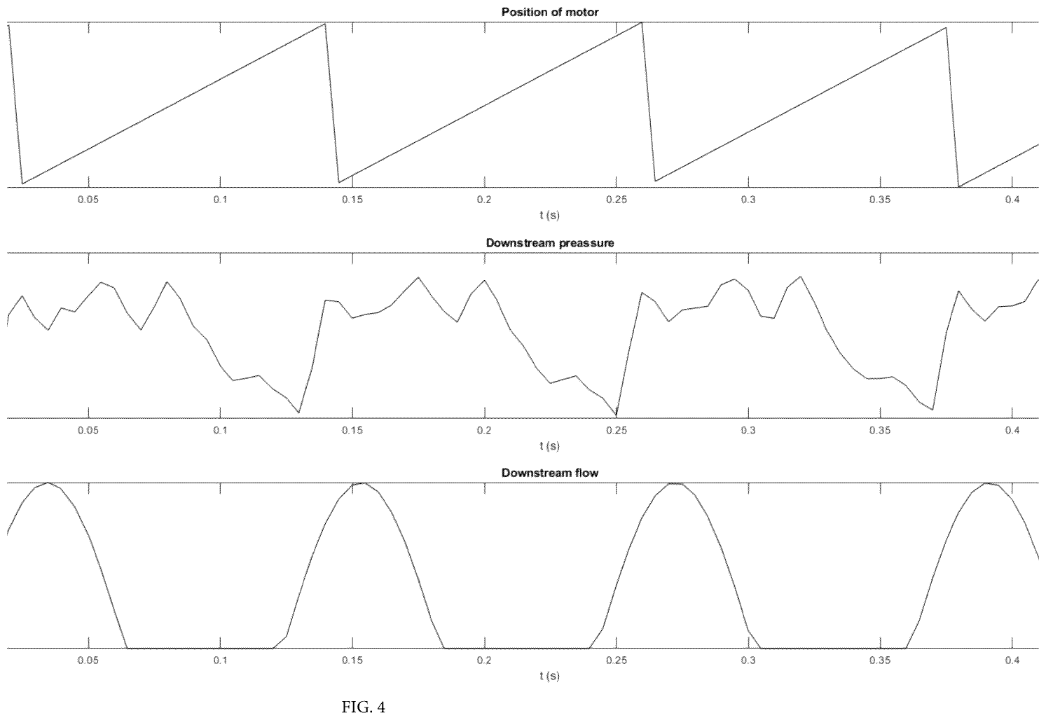

FIG. 4 provides plots that illustrate the open fluid path operational mode. The upper plot shows the relative position of the pump motor for PD fluid pump 70, where zero is an arbitrary start position of the pump motor measured in steps of a stepper motor having, in the illustrated example, 200 steps per revolution. The x-axis for each plot represents time. The middle plot shows the measured PD fluid pressure downstream from PD fluid pump 70. The lower plot shows the downstream PD fluid flow driving the pressure in the middle plot. The desired piston turning or home position corresponds to the point where PD fluid flow in the lower plot changes from greater than zero to zero.

In FIG. 4, the fluid path downstream of PD fluid pump 70 is relatively open with only limited frictional or other resistance, wherein the pressure varies depending on the location and orientation of piston 70p and indirectly the angular position of the pump motor, e.g., stepper motor. To be more precise, it is assumed that the dynamic flow to pressure relationship will be linear and of a lowpass character with some phase shift. In addition, there will be nonlinear effects of pressure reflections in system 10, which distort the linear dynamics, making it hard to extract features corresponding to the position of piston 70p in all cases. But since flow fluctuations occur at a relatively high frequency compared to the hydraulic compliance of the tubing of PD machine 20, the output of pressure sensor 78a, 78b1 or 78b2 will have a low pass character.

In FIG. 4, it is clear that the downstream PD fluid flow and pressure have both a linear dynamic relationship (change together) portion (corresponding to low flow rates) as well as a nonlinear (casual) relationship portion, including the downstream pressure remaining high long after PD fluid flow has stopped. The non-linear relationship portion is likely due to reflections in the PD fluid due to the flexible tubes or lines of PD machine 20. As can be seen in FIG. 4, however, the repeatable minimum downstream PD fluid pressure provides a useful point that control unit 100 uses to relate to a position of piston 70p (and corresponding angular position of the pump motor shaft. The minimum downstream pressure aligns rather well with the start (step zero of the stepper motor) of a piston push (partial/compression) stroke, which is also the piston turning position, which corresponds to where the PD fluid flowrate transitions from zero to positive. The minimum downstream pressure may be expected however to be phase shifted compared to the actual piston turning point. Control unit 100 is therefore programmed to look for a minimum pressure to identify the turning point of piston 70p, which can be related to the home position by a shift of 180 degrees with some compensation for phase shift of the minimum pressure. Control unit 100 is configured to iteratively determine the minimum pressure for each piston revolution. The pump position at the minimum pressure in each revolution is then used to update an average position of the minimum pressure. The use of the average position results in a more reliable position that can be related to the home position with a shift of 180 degrees and some phase shift depending on pump speed.

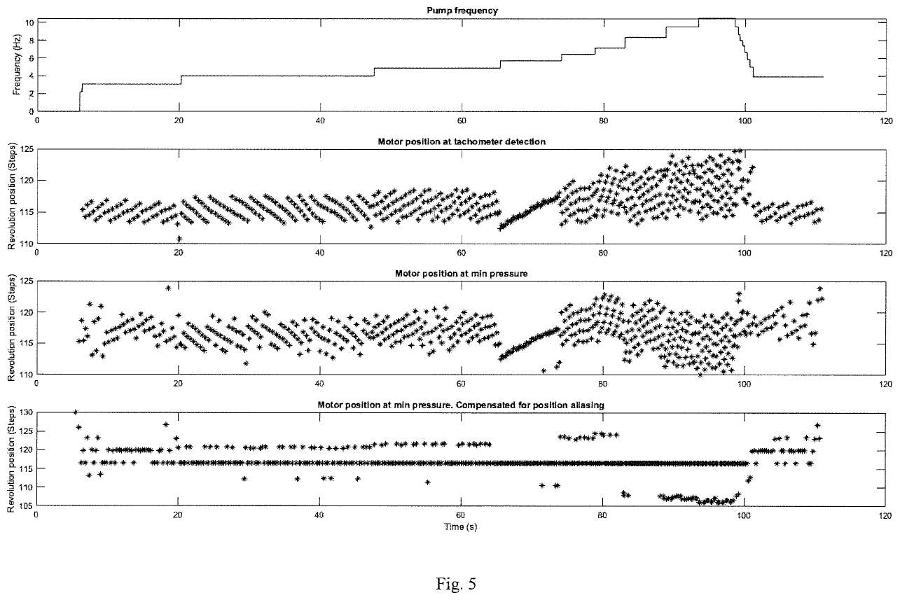

An example of the use by control unit 100 of the minimum downstream PD fluid pressure for detecting a location or position of piston 70p for different frequencies of PD fluid pump 70 is illustrated in FIG. 5. In FIG. 5, an optical tachometer is used to provide an accurate absolute position measurement for comparison purposes. The upper plot shows frequency of the pump motor overtime, which is varied for experimental purposes. The upper-middle plot shows the relative pump motor position at each tachometer tick per pump revolution collected over time at the different pump frequencies. The relative motor position is a number of steps between 0 and 200 (see FIG. 4 for reference). The lower-middle plot shows the relative motor position over each rotation using the minimum downstream pressure shown in FIG. 4. These are the values that are used to create an average of the home position in the current embodiment. The middle plots in FIG. 4 show that the tachometer tick motor position and the minimum downstream pressure motor position align well, yielding approximately the same position reading of 115 to 120 steps. The lower plot compensates for position aliasing of the tachometer readings in the upper-middle plot for comparison to the minimum downstream pressure readings in the lower-middle plot, leaving only the spread in position caused by inaccuracies in the pressure readings.

Use of the minimum downstream pressure readings to determine the desired piston turning or home position has the advantage that it can be used online, during treatment, to detect the minimum pressure and stop PD fluid pump 70 at the desired position. Also, the minimum downstream pressure detection involves taking many pressure measurements (high sample rate), and averaging the pressure measurements, which increases reliability.

### Closed Fluid Path Operational Mode

In the closed fluid path operational mode, system 10 stores an algorithm in control unit 100, which is used to find the piston turning or home position for piston 70p when it is needed for stopping PD fluid pump 70. Here, the fluid path within which the pressure is read is closed off via one of the valves illustrated in FIGS. 1 and 2. The fluid path may be closed at any time while system 10 is idle. The algorithm can be run at startup, wherein the determined home position is saved for later use. The algorithm may also be run at any point in which PD fluid pump 70 is to be placed in the home position. PD fluid pump 70 pumps PD fluid into the closed fluid path, causing a stationary pressure buildup. Assuming that the elasticity of the lines or tubes of PD machine 20 is linear, the pressure measured by one or more of pressure sensors 78a, 78b1 or 78b2 will increase linearly with inserted PD fluid volume. Since the displacement of piston 70p follows a sinewave, the downstream pressure likewise follows a sinewave. The pressure of a stroke as a function of time assuming a constant rotational speed or frequency of the motor of PD fluid pump 70 of 0.5 Hz (or other suitably slow speed to achieve good recursive least squares (“RLS”), while not being too slow to force high signal to noise) will therefore be:

$$p ( t ) = A ( 1 - cos ( π t ) ) where , 0 \leq t \leq 1$$

Assuming that piston 70p at time, t=0 is at the start of a piston stroke pushing fluid in the direction of one or more downstream pressure sensor 78a, 78b1, or 78b2. At time, t=1 the piston stroke is finished and piston 70p of PD fluid pump 70 is at the desired home position. Since a small amount of air in the system may exist so as to change tubing or line compliance in PD machine 20, A is not known, which means detection based on the equation above would not be reliable. It is contemplated therefore to use an algorithm based instead on the derivative of p(t):

$$\frac{dp ( t )}{dt} = A sin ( π t ) where 0 \leq t \leq 1$$

The desired piston turning or home position may be defined to be at t=0 (π rad from start position), which yields that the derivative is dp(1)/dt=A*sin(π)=0, which is used as a detection point or feature to find the relative position of the stepper motor at the home position. To do this reliably the derivative is filtered. In one embodiment, infinite impulse response (“IIR”) filters are not desirable because of their large phase delays. A finite impulse response (“FIR”) filter is a better option, but because the sample time needs to be very high in comparison to the frequency of PD fluid pump 70 to give a suitable resolution of the stepper motor position, the FIR filter would need to be of very high order to sufficiently smooth the signal enough.

In some embodiments, a Kalman filter/linear quadratic estimator (‘LQE’) can be used to filter the derivative sufficiently to detect a zero crossing without introducing phase delay. To do this, an auto regressive (“AR”) model of the underlying signal Asin(πt) is determined by the control unit 100. The AR model for a sine wave Asin(πt) is a second order process characterized by the following equation:

$$dp ( {t}_{k + 1} ) / dt = 2 cos ( 2 π ( Ts ) ω ) * dp ( {t}_{k} ) / dt - dp ( {t}_{k - 1} ) / dt$$

In the above equation, =tₖ₊₁−tₖ. It should be appreciated that the AR model does not have an amplitude component A. While the AR model has a specific frequency, the amplitude A will only depend on the initial values. As such, the AR model works for any amplitude A. This is a key feature of the AR model when the model is applied to the derivative of the pressure signal since A is unknown and may vary between runs. This feature gives the LQE filter the ability to adapt to any sinusoid with a specified frequency, which is known from the pump rotation frequency.

The AR model can be rewritten on state space form. Additionally, a state representing the measured pressure is introduced by adding an integrator over the pressure derivative from the AR model. It is then possible to formulate a LQE filter algorithm that uses pressure observations directly as input and provides a state representing a filter value of the pressure derivative, as provided below:

$${X}_{k} = [ \begin{matrix}dp ( {t}_{k} ) / dt \\ dp ( {t}_{k - 1} ) / dt \\ p ( {t}_{k} )\end{matrix} ] \qquad ( 1 - 1 )$$

$$F = [ \begin{matrix}2 cos ( π Ts ) & - 1 & 0 \\ 1 & 0 & 0 \\ Ts & 0 & 1\end{matrix} ] \qquad ( 1 - 2 )$$

$${H}_{1} = [ 001 ] \qquad ( 1 - 3 )$$

$${H}_{2} = [ 100 ] \qquad ( 1 - 4 )$$

$${\hat{X}}_{k \vert k - 1} = F {\hat{X}}_{k - 1 \vert k - 1} \qquad ( 1 - 5 )$$

$${P}_{k \vert k - 1} = {FP}_{k - 1 \vert k - 1} {F}^{T} + {R}_{1} \qquad ( 1 - 6 )$$

$${e}_{k} = {z}_{k} - {H}_{1} {\hat{X}}_{k \vert k - 1} \qquad ( 1 - 7 )$$

$${K}_{k} = \frac{{P}_{k \vert k - 1} {H}_{1}^{T}}{{R}_{2} + {H}_{1} {P}_{k \vert k - 1} {H}_{1}^{T}} \qquad ( 1 - 8 )$$

$${P}_{k \vert k} = {P}_{k \vert k - 1} - {K}_{k} {H}_{1} {P}_{k \vert k - 1} \qquad ( 1 - 9 )$$

$${\hat{X}}_{k \vert k} = {\hat{X}}_{k \vert k - 1} + {K}_{k} {e}_{k} \qquad ( 1 - 10 )$$

$$( {t}_{k} ) / dt = {H}_{2} {\hat{X}}_{k \vert k} \qquad ( 1 - 11 )$$

The LQE filter assumes the following of the true state, Xₖ₊₁, and observation of the true state, zₖ.

$${X}_{k + 1} = {FX}_{k} + {v}_{k} , where E ( {v}_{i} {v}_{j}^{T} ) = {R}_{1} for all i \neq j \qquad ( 1 - 12 )$$

$${z}_{k} = {H}_{1} {X}_{k} + {w}_{k} where E ( {w}_{i} {w}_{j}^{T} ) = {R}_{2} for all \neq j \qquad ( 1 - 13 )$$

In the above equations, E(·) is the mean operator and vₖ and wₖ are vectors of uncorrelated white noise. \hat{X}_{k|k-1} denotes the estimated a priori (predicted) state vector using the model specified by equation (5) without considering the pressure observations zₖ. \hat{X}_{k|k} denotes the posteriori state estimate given the observations, zₖ, up to time k. Similarly, P_{k|k} and P_{k|k-1} denote the a priori and posteriori covariance matrix estimates of the state vector.

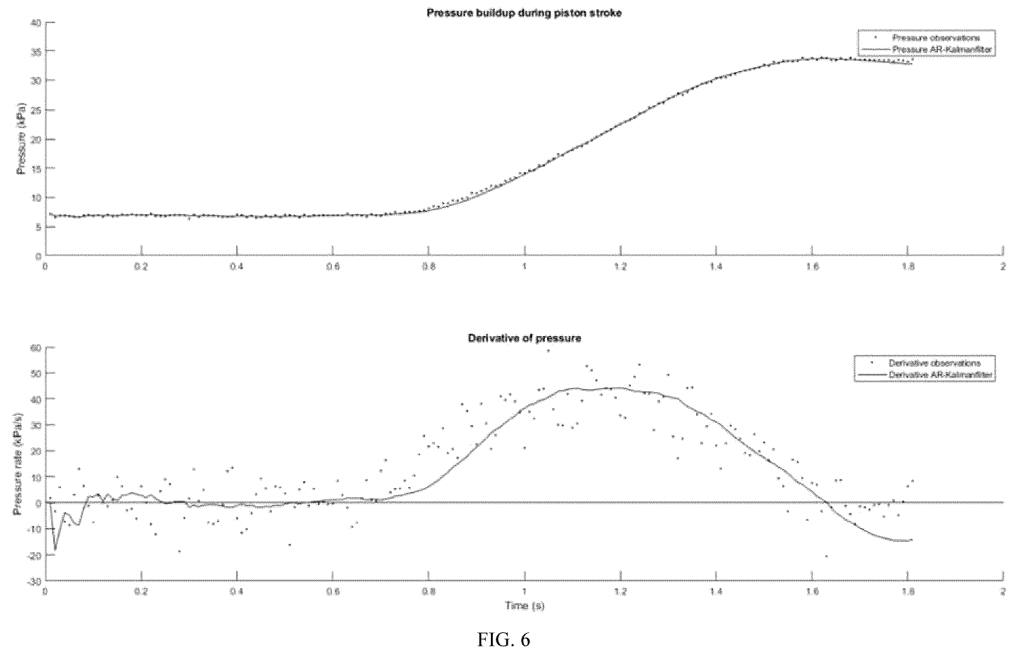

Given the above recursive algorithm specified by equation (1-11), the pressure derivative can be filtered by adjusting the process and measurement noise covariance matrices R₁ and R₂ to give a sufficiently hard filtering off the signal. To achieve this, the elements of the noise covariance matrices should have the following relation R₁<R₂. If elements of R₁ are too small, the filter will be slow at adapting to changes not described by the model. Since the objective is to filter a half sinusoid dp(t)/dt=max(0, Asin(2πt)) where all negative values are cut out while the model is assumed to be a full sinusoid, the transition between dp(t)/dt=0 and dp(t)/dt=Asin(2πt) will be slow. A tradeoff between filter level and adoption to this transition in the signal is inherent in the standard LQE filter by the relation between R₁ and R₂. Alternatively, the transition is detected, and the filter is reset, which could allow even more filtering of the signal by the control unit 100. An example of using the recursive algorithm shown in equation (1-11) above where a tradeoff in the R₁<R₂ relation has been tuned for the specific case can be seen in FIG. 6.

As shown in FIG. 6, there is a defined relationship between filtered pressure and measured pressure observations zₖ. The second graph shows the filtered derivative of pressure dp(t)/dt given by equation 1-11 above using the LQE filter algorithm. The graph also shows the numerically calculated derivative of the pressure with a FIR filter for reference. There is a temporary difference between the filtered values and pressure observations at time t=0.8 s. This is a transient of the LQE filter when adopting to the transition between dp(t)/dt=0 and dp(t)/dt=Asin(2πt), which is not modeled. This transient has faded away at the point of the zero crossing at t=1.65 s, which is the point of interest.

There are many ways the above outlined method can be varied to adapt an appropriate filter. The model of the underlying function as well as the observer/filter may be varied independently. Another option can include applying a polynomial model with recursive least square (“RLS”) algorithm to the underlying function Asin(πt), which is updated iteratively during the detection. The polynomial fit is less accurate at 0 rad but becomes accurate at the end of the stroke at π rad, where the polynomial fit will have converged enough to yield a “filtered” value of the pressure derivative. The polynomial fit requires more processing computation at control unit 100 than say an FIR filter but needs less saved data and has no phase delay if an appropriate polynomial is used. For the RLS algorithm, assume for a noisy observation of dp(t)/dt:

$${y}_{k} = \frac{dp ( {t}_{k} )}{dt} + {v}_{k}$$

where vₖ is assumed to be white noise, that dp(tₖ)/dt can be modeled with the polynomial:

$$dp ( {t}_{k} ) / dt = {ϕ}_{k} θ = [ {t}_{k} {t}_{k}^{2} {t}_{k}^{3} {t}_{k}^{4} ] [ \begin{matrix}{θ}_{1} \\ {θ}_{2} \\ {θ}_{3} \\ {θ}_{4}\end{matrix} ]$$

which results in the following recursive least squares (“RLS”) algorithm involving equations (1) to (6) that are stored in control unit 100 of PD machine 20:

$${ϕ}_{k} = [ {t}_{k} {t}_{k}^{2} {t}_{k}^{3} {t}_{k}^{4} ] \qquad ( 2 - 1 )$$

$${e}_{k} = {y}_{k} - {ϕ}_{k} \qquad ( 2 - 2 )$$

$${K}_{k} = \frac{{P}_{k - 1} {ϕ}_{k}^{T}}{λ + {ϕ}_{k} {P}_{k - 1} {ϕ}_{k}^{T}} \qquad ( 2 - 3 )$$

$${P}_{k} = \frac{{P}_{k - 1} - {K}_{k} {ϕ}_{k} {P}_{k - 1}}{λ} \qquad ( 2 - 4 )$$

$$= + {K}_{k} {e}_{k} \qquad ( 2 - 5 )$$

$$dp dt = {ϕ}_{k} \qquad ( 2 - 6 )$$

In the above equations (1-1) to (1-6), the matrix Pₖ is a covariance matrix, which is of size 4×4 and is positive and definite (implying that it is symmetric). Pₖ and the coefficients, [symbol] , are saved variables between iterations of the algorithm above. The “hat” (\hat{ }) denotes an estimate of the underlying coefficients θₖ, wherein the subscript k is the iteration number. The symbol λ is a “forgetting factor” weight, which puts a higher weight on recent samples in the RLS estimate. The weight λ=1 provides equal weight to all samples. The weight λ<1 provides more weight to recent samples. An approximation is that 1/(1−λ) samples will be stored in or more memory device 104. The speed of the motor of PD fluid pump 70, such as 0.1 to 10 revolutions per minute (“rpm”), is chosen to be fast enough to obtain a high enough derivative signal (signal to noise) and at the same time slow enough to obtain a sufficient resolution of the relative position of the pump motor for the relevant sample time.

For the RLS estimate to provide good results, it is important that time to =0 aligns well with the start of a piston stroke, so that a full stroke of piston 70p (0 to π rad) is observed by the RLS algorithm. The full observation is achieved by resetting the RLS algorithm if the estimate value dp [symbol] /dt is less than 0. Resetting the algorithm is performed by setting tₖ=0 and reinitializing Pₖ and [symbol] . When the PD fluid pressure starts to rise, the RLS is updated by control unit 100 to provide a function fit. Control unit 100 continues to update the RLS estimate of dp(tₖ)/dt until the estimate drops below zero, which is the detection point for home position.

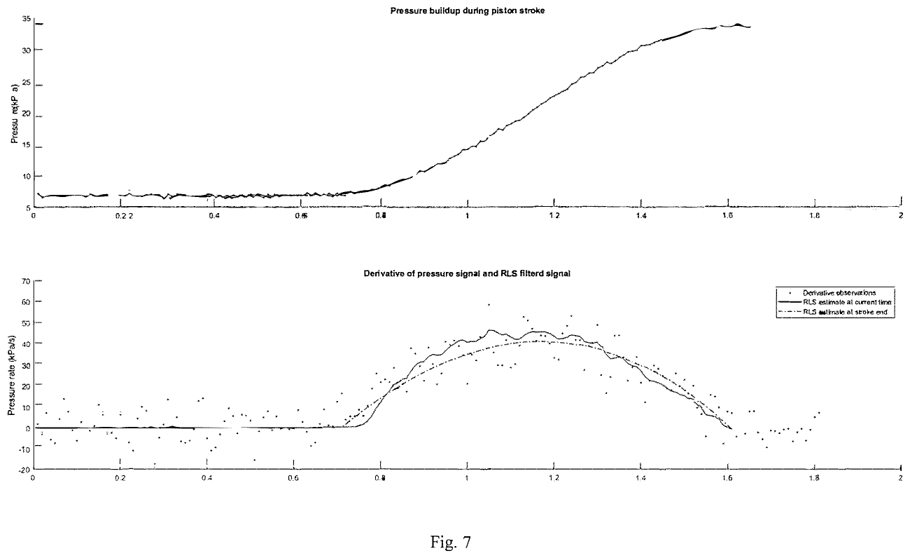

FIG. 7 illustrates an example outcome using the above RLS algorithm (including equations (1) to (6)) over the x-axis, which is time. In FIG. 7, the top plot shows the fluid pressure measured by one or more pressure sensor 78a, 78b1, or 78b2 in a closed fluid path during one full stroke of PD fluid pump 70. The bottom plot shows the derivative of the pressures in the top plot as dots. In the bottom plot, the solid line shows the RLS estimate at current time, while the dashed line shows the RLS estimate for the past stroke after the stroke is finished (zero crossing detected). The cross symbol in FIG. 7 marks the zero crossing. The zero crossing of the dashed line in the lower plot of FIG. 7 and indicates the piston turning or home position of piston 70p within cylinder 70c.

Control unit 100 of PD machine 20 of system 10 is in one embodiment programmed to store equations (1-1) to (1-13) and/or (2-1) to (2-6), to receive pressure measurements from one or more downstream pressure sensor 78a, 78b1, or 78b2, to filter and analyze the derivative of the pressure readings, and to stop the pump piston 70p when the derivative pressure signal reaches a zero crossing. At such a time, pump piston 70p is at or near the turning or home position.

It is contemplated as an improvement to the RLS algorithm in which additional sensor readings are used to update the algorithm so as to be based on a Kalman/linear quadratic estimation (“LQE”) filter, which fuses multiple pressure sensor readings to better suppress largely uncorrelated noise. The equations below are for a LQE filter operating with two or more pressure sensors 78a, 78b1 or 78b2 that measure PD fluid pressure along a same downstream fluid path. The readings of the two or more pressure sensors help to suppress uncorrelated noise:

$$ϕ = [ \begin{matrix}t & {t}^{2} & {t}^{3} & {t}^{4} \\ t & {t}^{2} & {t}^{3} & {t}^{4}\end{matrix} ] \qquad ( i )$$

$${e}_{k} = {y}_{k} - ϕ \qquad ( ii )$$

$${K}_{k} = \frac{{P}_{k - 1} {ϕ}_{k}^{T}}{{R}_{2} + {ϕ}_{k} {P}_{k - 1} {ϕ}_{k}^{T}} \qquad ( iii )$$

$${P}_{k} = {P}_{k - 1} - {K}_{k} {ϕ}_{k} {P}_{k - 1} + {R}_{1} \qquad ( iv )$$

$$= + {K}_{k} {e}_{k} \qquad ( v )$$

$$dp dt = {ϕ}_{k} , \qquad ( vi )$$

The above equations (i) to (vi) are stored in control unit 100 and enable noise suppression by using more than one sensor 78a, 78b1, or 78b2. Depending on the configuration of sensors 78a, 78b1, or 78b2, equations (i) to (vi) may be modified to capture the relationship between the sensor readings.

Each of the pressure sensing methods discussed herein is configured to suppress noise on a pressure signal from one or more pressure sensor 78a, 78b1, or 78b2. In any scenario, however, the primary limiting factor for accurately finding the piston turning or home position is the sample rate of one or more processor 102 of control unit 100 together with the rotational speed of the pump motor for PD fluid pump 70. For higher rotational pump speeds, e.g., during treatment for the open fluid path operational mode, greater sample rates by one or more processor 102 will be needed. An advantage of the closed fluid path operational mode is that the pump motor may be run at a slow rotational speed, which lessens the need for a high sample rate. It is also contemplated in the open fluid path operational mode, for control unit 100 to cause PD fluid pump 70 at or near the end of a patient fill or dwell to slow the speed of the PD fluid pump considerably, e.g., so as to reduce the flowrate to between zero and fifty milliliters (“ml”) per minute, so that a lesser sample rate by processor 102 still yields an accurate turning point or home position for PD fluid pump 70.

In an example embodiment, processor 102 is configured to have a sample rate of 50 to 500 Hz for any version of the open fluid path operational mode of the present disclosure, and likewise 50 to 200 Hz for any version of the closed fluid path operational mode of the present disclosure.

### CONCLUSION

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. In various examples, PD system 10 does not have to use redundant or durable components, and may instead employ a disposable set having a disposable pumping portion that contacts the corresponding medical fluid. In another example, while disposable filter set 40 would not be needed as a last chance filter for a system not having heat disinfection, disposable filter set 40 may still be provided if the fresh PD fluid is made online at the time of use as a last chance filter for the online PD fluid.

In another example, PD fluid pumping with the disposable set may be performed alternatively via peristaltic pump actuation of a pumping tube segment provided with the disposable set. That is, while the pump actuator for PD fluid pump 70 is illustrated herein as actuating a piston 70p, system 10 may be applied to other types of pump actuators, such as peristaltic pump actuators. Here, the pressures corresponding to positions of the other types of pump actuators may be determined empirically or via an algorithm as illustrated above, and then programmed into control unit 100, which operates the motor driver 108 of the other type of pump actuator. Moreover, while the pump motor for PD fluid pump 70 is described herein as being a stepper motor, system 10 may be applied to other types of pump motors, such as AC or DC brushed or brushless motors, and the like, but wherein such alternative motors operate with a rotational position detection device, such as an incremental or absolute encoder, the output of which is linked to the angular position of pump piston 70p in the same manner as the steps of a stepper motor as described herein.

The invention is claimed as follows:

1. A peritoneal dialysis (“PD”) system comprising:
a PD fluid pump including a reciprocating member having a home position;
at least one pressure sensor positioned and arranged to sense the pressure of PD fluid pumped by the PD fluid pump; and
a control unit configured to control the PD fluid pump and to take PD fluid pressure readings from the at least one pressure sensor, the control unit further configured to use the PD fluid pressure readings to determine when the reciprocating member is in the home position, and when desired to stop the PD fluid pump when the reciprocating member is in the home position.

2. The PD system of claim 1, wherein the at least one pressure sensor is positioned and arranged to take the pressure of PD fluid along a line downstream of the PD fluid pump.

3. The PD system of claim 1, wherein the at least one pressure sensor is positioned and arranged to take the pressure of PD fluid along an open fluid line, wherein the pressure of PD fluid changes in a pattern according to movement of the reciprocating member, and wherein a feature in the pattern is related the home position.

4. The PD system of claim 3, wherein the open fluid line is used during a PD treatment, the at least one pressure sensor taking the pressure of PD fluid during the PD treatment.

5. The PD system of claim 3, wherein the control unit is configured to take PD fluid pressure readings from the at least one pressure sensor at a sample rate of 50 to 500 Hz.

6. The PD system of claim 1, wherein the at least one pressure sensor is positioned and arranged to take the pressure of PD fluid along a closed fluid line, wherein the control unit is configured to use at least one algorithm to analyze a derivative of a change of the pressure of PD fluid over a stroke of the PD fluid pump, and wherein a zero crossing of the derivative of the change of the pressure of PD fluid over the stroke corresponds to the home position.

7. The PD system of claim 6, wherein the closed fluid line is closed when the system is in an idle state, wherein the control unit uses the at least one algorithm.

8. The PD system of claim 6, wherein the control unit is configured to take PD fluid pressure readings from the at least one pressure sensor at a sample rate of 50 to 200 Hz while the control unit uses at least one algorithm.

9. The PD system of claim 6, wherein the control unit is configured to cause the PD fluid pump to pump at a rate of 0.1 to 10 rpm while the control unit uses at least one algorithm.

10. The PD system of claim 6, wherein the at least one algorithm includes a recursive estimation algorithm, optionally a recursive least squares (“RLS”) algorithm or a linear quadratic estimator (“LQE”) algorithm.

11. The PD system of claim 6, wherein the at least one pressure sensor includes a plurality of pressure sensors positioned and arranged to take the pressure of PD fluid along the closed fluid line, and wherein the at least one algorithm employs a linear quadratic estimation (“LQE”) filter.

12. The PD system of claim 1, wherein the PD fluid pump is a piston pump and the reciprocating member includes a piston moving within a cylinder.

13. The PD system of claim 12, wherein the home position corresponds to a distal end of the piston being closest to an end wall of the cylinder.

14. A peritoneal dialysis (“PD”) system comprising:
a PD fluid pump including a reciprocating member having a home position;
at least one pressure sensor positioned and arranged along an open fluid line to sense the pressure of PD fluid pumped by the PD fluid pump, wherein the pressure of PD fluid changes in a pattern according to movement of the reciprocating member; and
a control unit configured to control the PD fluid pump and to take PD fluid pressure readings from the at least one pressure sensor, the control unit further configured to use a feature in the pattern to determine when the reciprocating member is in the home position.

15. The PD system of claim 12, wherein the feature includes a minimum pressure in the PD fluid pressure pattern.

16. A peritoneal dialysis (“PD”) system comprising:
a PD fluid pump including a reciprocating member having a home position;
at least one pressure sensor positioned and arranged along a closed fluid line to sense the pressure of PD fluid pumped by the PD fluid pump; and
a control unit configured to use at least one algorithm to analyze a derivative of the pressure of PD fluid over a push partial stroke or pull partial stroke of the PD fluid pump, and wherein a zero crossing of the derivative of the change of the pressure of PD fluid over the stroke corresponds to the home position.

17. The PD system of claim 16, wherein the at least one algorithm includes a recursive estimation algorithm, optionally a recursive least squares (“RLS”) algorithm or a linear quadratic estimator (“LQE”) algorithm.

18. The PD system of claim 16, wherein the at least one pressure sensor includes a plurality of pressure sensors positioned and arranged to take the pressure of PD fluid along the closed fluid line, and wherein the at least one algorithm employs a linear quadratic estimation (“LQE”) filter.
